(19) United States
(12) Patent Application Publication
(10) Pub. No.: US 2015/0045012 A1
(43) Pub. Date: Feb. 12, 2015

(54) OPHTHALMIC ADAPTER FOR PERSONAL ELECTRONICS

(71) Applicant: Neuroptics, Inc., Irvine, CA (US)

(72) Inventor: Kamran Siminou, Newport Coast, CA (US)

(21) Appl. No.: 14/454,721

(22) Filed: Aug. 8, 2014

Related U.S. Application Data
(60) Provisional application No. 61/863,431, filed on Aug. 8, 2013.
(60) Provisional application No. 61/868,581, filed on Aug. 22, 2013.

(51) Int. Cl. H04M 1/725; A61B 3/00; H04W 4/00

(52) U.S. Cl. CPC H04M 1/72527; H04W 4/008; H04M 1/72525; A61B 3/00
USPC 455/419; 455/41.2

(57) ABSTRACT

A mobile phone cover having a housing capable of receiving a mobile telephone, wherein the housing comprises a telecentric lens, one or more IR LEDs, one or more visible LEDs, and means for communicating digital information or commands between the mobile phone cover and a mobile phone.

### BACKGROUND

This application claims the benefit of priority to the following provisional applications each of the above-identified applications are incorporated herein by reference in their entirety:

Ser. Nos. 61/863,431, filed Aug. 8, 2013 and 61/868,581, filed Aug. 22, 2013.

This disclosure relates to functional covers for personal electronic devices that have built-in cameras, such as mobile phones, and tablet computers, such as the Apple Ipad® and Samsung Galaxy tablet computers. More specifically, it relates to covers that have ophthalmic instrumentation functionality and are sized and designed to receive a personal electronic device having a built-in camera, such as a mobile phone or a tablet computer. More specifically, it relates to covers that have a built-in telecentric lens and corresponding electronics that can be electronically coupled to a mobile phone or tablet computer that has a built-in camera.

Mobile phones and tablet computers with built-in cameras have become ubiquitous in modern society. They can be used not just for telephonic communication but for hundreds and thousands of other purposes and uses as a result of software applications that can be used with these devices. There are applications for almost every purpose conceivable.

One such application is to use a cell phone or tablet computer as an ophthalmic instrument, i.e., an instrument that can be used to image a human or animal subject's eye or eyes and provide the user with information about the eye(s), such as the eye(s) static characteristics or its dynamic response to a stimulus, such as a light stimulus. Such applications can be used for security purposes, such as biometric applications that are used to identify a subject by his or her unique ocular characteristics, such as the unique pattern of the subject's iris or sclera/iris border. Another application is to use the camera of the mobile phone or tablet for medical diagnostic purposes to monitor or check a subject's eye(s) to diagnose a medical condition.

In one such application, the camera of mobile phones can be used to image a pupil of an eye(s) and to provide or analyze pupilometry data. Currently, very precise pupillary measurements can only be taken with highly specialized pupilometers, such as those provided by Neuroptics, Inc. Examples include the NeurOptics® NPi 100, the VPi® 200, the PLR® 200 and the PLR® 2000. These devices are highly specialized pupilometry systems that are used by professionals. The problem is that they are less accessible to the general public and they are not always available on hand to assess a subject in an emergency situation or a situation where an assessment of the subject's pupils may be critically important for short-term or long-term prognosis of the subject. These devices are not ubiquitous like mobile phones and tablet computers.

Unfortunately, although mobile phones and tablet computers are ubiquitous, they cannot be used to conduct reasonably precise pupilometry. Pupilometry applications using a mobile phone's or tablet computer's own camera are not possible. That's because those cameras are not adapted to be able to conduct on the fly-measurements to adjust for distance between the camera and the pupil, and such measurements are necessary for conducting precise measurements of the pupil.

Thus, what is needed are adapters with specialized lenses, which can be removably coupled to a mobile phone or pupilometer that have cameras. Such adapters can provide a useful application for clinicians who currently use standard imprecise techniques for pupillary assessment. It can also be used by emergency medical technicians during emergency situations to quickly assess the status of a patient during an emergency situation using their own mobile phones or tablet computers. It can even be used by coaches, team doctors, referees, school nurses, teachers, parents and the general public so that they can use their mobile phones or tablet computers to assess minor or major traumatic brain injuries during sporting events, school activities, and the like, particularly to quickly and accurately assess the level of traumatic brain injury or concussion suffered by an individual.

Given the large number of concussions suffered in both professional and amateur team sports in the United States alone, there is a tremendous need for adapters that can transform a mobile phone or tablet computer into a reasonably precise and reasonably priced pupilometer. For example, the NFL was just recently sued by over 5000 former players and their surviving family members for concussions that they suffered, which lead to permanent brain damage and a condition called chronic traumatic encephalopaphy (“CTE”). This condition is believed to be caused by repeated concussions, which is why it is so important to diagnose a concussion in real-time when it happens in order to take proper precautions to minimize the risk of a second concussion. There is therefore a need for adapters that can transform a mobile phone or tablet into a pupilometer that can be used by field doctors, coaches, referees and even parents and spectators to quickly and easily assess the neurological condition of the player by assessing his or her pupillary response.

There is also a need for adapters that can be applied to mobile phones and tablets to transform those devices into security screening equipment to screen and identify people for security purposes. Such devices can be used to image a pupil's eye, i.e., take biometric data from the subject, and identify the subject.

### SUMMARY

In accordance with one embodiment, described is a mobile phone cover having a housing capable of receiving a mobile telephone, wherein the housing comprises a telecentric lens, one or more IR LEDs, one or more visible LEDs, and means for communicating digital information or commands between the mobile phone cover and a mobile phone.

In accordance with another embodiment, a pupilometry system includes a mobile phone cover and a computer program. The mobile phone cover has a housing capable of receiving a mobile telephone, wherein the housing comprises a telecentric lens, one or more IR LEDs, one or more visible LEDs, and means for communicating digital information or commands between the mobile phone cover and a mobile phone. The computer program can be downloaded onto a mobile phone for controlling the operation of the mobile's phone's camera, the telecentric lens, the one or more IR LEDs, and the one or more visible LEDs.

### BRIEF DESCRIPTION OF THE DRAWINGS

FIG. 1 is a perspective view of a mobile phone cover with pupilometry components, including a telecentric lens, visible LEDs, IR LEDs, an IR filter, and supporting electronics in accordance with one embodiment.

FIG. 2 is another perspective view of the mobile phone cover depicted in FIG. 1.

FIG. 3 is a perspective view of a mobile phone housed within the mobile phone cover depicted in FIG. 1.

### DETAILED DESCRIPTION

Provided here is a cover for a mobile phone or tablet computer that has a telecentric lens. Telecentric lenses were discovered more than a century ago, and various patents relating to them have been in existence for decades. (See for example, U.S. Pat. No. 2,600,805, which is incorporated herein by reference). A telecentric lens by definition limits the angular acceptance (from object plane) or exitance (to image plane) or both (double telecentric). The smaller the chief ray acceptance-angle of the lens the better the telecentric performance. This limitation fo chief ray acceptance angle is accomplished by the strateig placement of an aperture stop exactly at one compounded focal distance behind the group of lenses in front of the group of lenses for image plane example. For the double telecentric ase, both front and rear lens groups are placed exactly one focal length (compounded separately for each of the front and rear group) from the aperture stop.

In general, lenses with acceptance angles of 1° or less are considered to be operationally telecentric. The chief ray originating from any point along the object is geometrically limited to be perpendicular to the object plane, which results in an invariant magnification for all object distances (for object side telecentricity). The range of practical operational object distance is limited by defocus/blur as the object is moved away from the preferred conjugate position.

One type of telecentric lens that is used in the invention is a double telecentric lens, placed over the fixed lens, rendering both the distance from the object to lens (new-attachment) and distance from attachment to internal fix lens (mobile phone camera lens) insensitive to distance of separation in terms of magnification error. In other words, the magnification of the pupil image projected tot eh sensor would depend neither on the distance of the pupil from the objective lense or the distance of the objective lens to the phone-camera lens. In addition, the use of an intensified array (e.g., night vision element) with front-end telecentric optics where the output screen is reimaged to the camera lens is also contemplated herein.

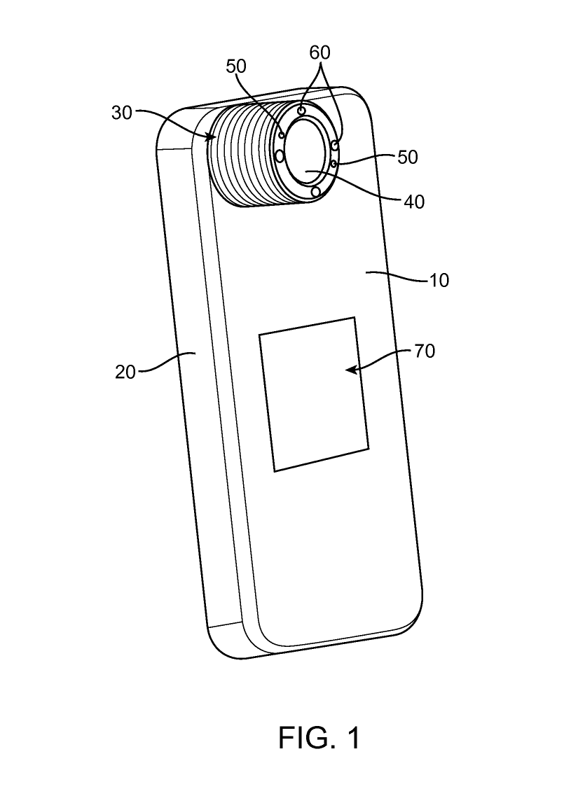

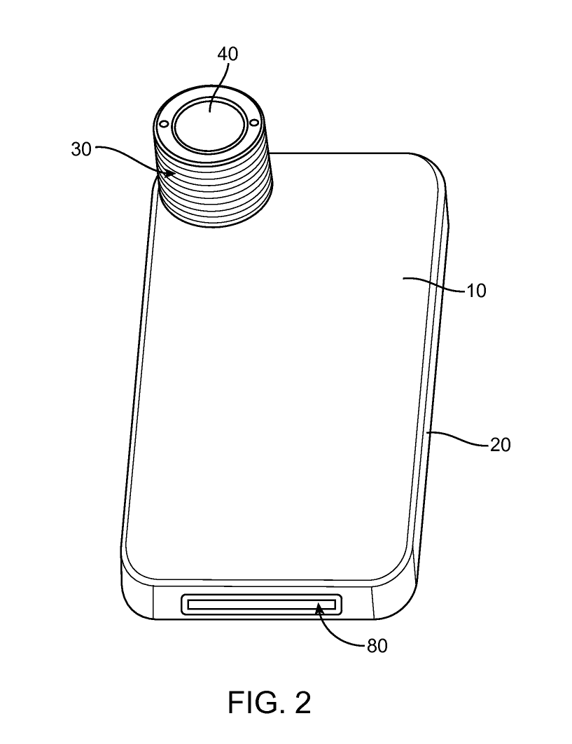

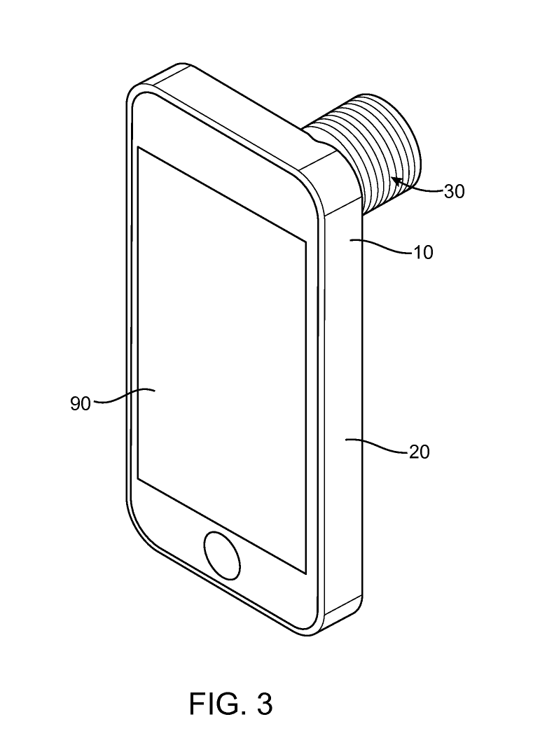

With the above in mind, one embodiment of a cover having a telecentric lens (from object plane, image plane, or doubly telecentric) is shown in FIGS. 1-3. Cover 10 has a rectangular shape that matches the shape of many mobile phones, or it can be adapted to match the shape of a particular mobile phone brand and model so that the housing 20 of the cover 10 mates with the mobile phone by receiving the mobile phone. FIG. 3 shows a mobile phone 90 inserted into the housing 20 in the conventional manner. The housing is flexible enough to flex to allow the mobile phone to be inserted into the housing, while at the same time being rigid and strong enough to protect the mobile phone from minor shocks or jarring. The cover 10 is actually a pupilometry adapter, because it transforms a mobile phone 90 into a pupilometer.

The cover 10 includes a telecentric lens 30. The telecentric lens 30 can be an object plane lens, an image plane lens, or a doubly telecentric lens. The telecentric lens has an IR filter 40, IR LEDs 50, and visible LEDs 60. It also includes electronics 70 that enables it to communicate with the phone, such as a electric circuit to power on and off the IR LEDs 50 and visible LEDs 60, as well as the aperture of the lens 30 itself The cover 10 can also have a light meter to measure ambient light. The electronics of the cover can communicate with the phone through a hard wire connection, such as a USB connection or other hardwire connection, or through a wireless connection, such as Bluetooth or RFID. The electronics 70 of the cover can include a power source, such as an internal batter, or it can be powered by the phone's power source. Conversely, if it has its own power source, it can be a source of backup power for the phone.

The telecentric lens 30, IR filter 40, IR LEDs 50 and visible LEDs 60 are all on the back side of the housing 20. The front side of the housing 20 forms a receptacle that receives the mobile phone 90. The bottom edge of the cover 20 has a communication connector 80 that can be connected to the mobile phone 90 so that the mobile phone 90 and cover 10 are in electrical and digital communication with one another when the connector 80 is connected to the mobile phone 90. For example, the connector 80 can be a USB connector connecting to the mobile phone device's USB port. Alternatively, the cover 10 can have an RFID tag that communicates with an RFID reader in the mobile phone 90, or Bluetooth technology that allows the cover 10 to communicate wirelessly with the mobile phone 90. Accordingly, the cover 10 communicates with the phone 90 so that the buttons and electronics on the phone can control the aperture and lens of the telecentric lens 30 as well as the activation of the IR LEDs 50 and visible LEDs 60

In an alternative embodiment, the lens 30 is not itself a telecentric lens, but when combined with the phone's built-in camera lens, the lens 30 acts a telecentric lens. In other words, the combination of the lens 30 and the lens of the built-in camera phone acts acts as a telecentric lens, such that the lens 30 is designed to work with the lens of the built-in camera phone to become a telecentric lens.

The cover 10 can be associated with a mobile phone pupilometry application that can be downloaded onto the mobile phone for operating the cover 10. When the phone is inserted into the housing 20, the application can be automatically launched. The mobile phone application can be designed to modify the operation of the mobile phone's built-in camera. The telecentric lens 30 of the cover fits over the lens of the built-in camera of the mobile phone. Using the pupilometry application, the mobile phone can be used to image a subject's pupil. The telecentric lens 30, visible LEDs 60 and IR LEDS 50 are controlled using the pupilometry application downloaded onto the mobile phone. The pupilometry application allows the buttons of the phone to be used to image a pupil using the camera's built-in camera and the telecentric lens 30 of the cover 10. For example, the built-in camera of the mobile phone can be activated to begin recording the a video of the pupil through the telecentric lens 30. Upon pressing a button on the mobile phone camera or pupilometry application graphical user interface displayed on the mobile phone display, the IR LEDs 50 are activated for a period of time (e.g., between 10 msec and 5 seconds) followed by a flash of light, or sequence of flashes of light, delivered by the visible LEDs 60, while the camera is recording the pupil through the telecentric lens 30. There can be one, two, three, four or more visible LEDs 60, and one, two, three, four or more IR LEDs 50. The IR LEDs 50 illuminate the pupil so that the pupil can be imaged by the built-in camera of the mobile phone. The visible LEDs 60 are used to provide a flash of white or yellow visible light to stimulate the pupils and cause a pupillary reaction to the flash of light or sequence of flashes of light.

The telecentric lens 30 is important to the operation of a pupilometer by a mobile phone or a tablet computer for the following reason. Most lenses exhibit varying magnification for objects at different distances from the lens. This causes several problems for a pupilometry application used on a typical built-in camera of a mobile phone: the apparent size of objects changes with distance from the camera; some features or portions of the pupil may be hidden by objects that are closer to the lens, such as eyelashes or eyelid; the apparent shape of the pupil varies with distance from the center of the field of view (FOV); and objects appearing close to the edges are viewed from an angle, while objects near the center of the FOV are viewed frontally (circles near the center of the FOV become egg-shaped when moved towards the periphery). Most importantly, without a telecentric lens, a typical built-in camera doesn't have the ability to control for the size of the pupil no matter what the distance of the camera lens from the pupil. Moreover, without a headrest, mobile phones cannot be reliably kept at an exact distance without any movement while the pupil is being recorded. Thus, with the typical built-in camera of a mobile phone or tablet computer, the pupillary data is imprecise and corrupted, because the actual size of the pupil is not known and can appear to change with the movement of the phone relative to the subject's pupil.

Telecentric lenses, on the other hand, provide an orthographic projection, providing the same magnification at all distances. An object that is too close or too far from the lens may still be out of focus, but the resulting blurry image will be the same size as the correctly-focused image would be. Because their images have constant magnification and geometry, telecentric lenses are used for metrology applications, when a machine vision system must determine the precise size of objects independently from their position within the FOV and even when their distance is affected by some degree of unknown variations.

Thus, a telecentric lens fitted over the built-in camera of the phone or tablet computer provides a means for accurately measuring the actual size of the pupil and controlling for that size even as the mobile phone moves relative to the subject's pupil.

While the invention is susceptible to various modifications and alternative forms, specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the appended claims.

I claim:

1. A mobile phone cover comprising:
a housing capable of receiving a mobile telephone, wherein the housing comprises a telecentric lens, one or more IR LEDs, one or more visible LEDs, and means for communicating digital information or commands between the mobile phone cover and a mobile phone.

2. A pupilometry system comprising:
a mobile phone cover comprising a housing capable of receiving a mobile telephone, wherein the housing comprises a telecentric lens, one or more IR LEDs, one or more visible LEDs, and means for communicating digital information or commands between the mobile phone cover and a mobile phone;
a computer program that can be downloaded onto a mobile phone for controlling the operation of the mobile's phone's camera, the telecentric lens, the one or more IR LEDs, and the one or more visible LEDs.
